(19) United States
(12) Patent Application Publication
(10) Pub. No.: US 2014/0172062 A1
(43) Pub. Date: Jun. 19, 2014

(54) MEDICAL DEVICE HAVING A COVERING MATERIAL AND AN ELECTRIC STIMULATOR

(71) Applicant: Yong Soon Yoon, Jeonbuk (KR)

(72) Inventor: Yong Soon Yoon, Jeonbuk (KR)

(73) Assignee: YESU HOSPITAL MANEGEMENT FOUNDATION, Jeonbuk (KR)

(21) Appl. No.: 14/233,745

(22) PCT Filed: Jun. 25, 2012
(86) PCT No.: PCT/KR2012/004988

§ 371 Date: Jan. 18, 2014

(30) Foreign Application Priority Data
Jul. 20, 2011 (KR) 10-2011-0071962

(51) Int. Cl. A61N 1/04

(52) U.S. Cl. CPC A61N 1/0492
USPC 607/149

(57) ABSTRACT

The present invention provides a medical device having a covering material and an electrical stimulator. The electrical stimulator includes a main body which generates electrical stimulation and a pad which is detachably arranged at a damaged part of a living body and transfers the electrical stimulation generated by the main body to the damaged part. The covering material is disposed on a lower surface of the pad and comes into contact with the damaged part, protecting and treating the damaged part.

### CROSS REFERENCE TO RELATED APPLICATIONS AND CLAIM OF PRIORITY

This patent application is a National Phase application under 35 U.S.C. §371 of International Application No. PCT/KR2012/004988, filed 25 Jun. 2012, which claims priority to Korean Patent Application No. 10-2011-0071962 filed 20 Jul. 2011, entire contents of which are incorporated herein by reference.

### BACKGROUND

1. Field of the Invention

The present invention relates to a medical device that can treat a damaged part of a living body as well as electrically stimulate the living body.

2. Description of the Related Art

The skin is the natural outer layer of tissue that covers a living body. When the skin is severely damaged, the injury is likely to be fatal. When a living body has a damaged part such as a wound, a burn, or an injury, the damaged part is naturally healed. In this case, a wound covering material is used to effectively treat the damaged part of the living body and promote healing of the damaged part.

The wound covering materials should have excellent biocompatibility so as not to cause immunological rejection in damaged part. Additionally, they should sufficiently absorb blood, plasma, and other fluids exuded from the damaged part, and not generate toxic materials during degradation thereof when portions of the wound covering materials sink into skin tissue.

The wound covering materials need to come into appropriate contact with the damaged part of a living body and they need to prevent the damaged part from being infected. Yet additionally, they need to exhibit low reactivity to chemicals used to speed healing of the damaged part.

Conventional wound covering materials are made from various substances to have improved treatment effects on the wound. In the early stage of development of wound coverings, non-degradable polymers such as polytetrafluoroethylene, cellulose, acetate, silicone rubber, and polyurethane were used. However, such non-degradable materials have the drawback of partially sinking into skin tissue and permanently remain in the skin tissue. In order to overcome this drawback, wound dressings using a highly biocompatible material such as chitosan have been developed recently.

Specifically, Korean Patent Application Publication No. 2002-0075539 proposes a wound covering material using chitosan oligomer and electrolytes. However, this wound covering material has the drawback of being unable to physically defend against germs entering from the outside.

Furthermore, since chitosan oligomer is water-soluble, chitosan oligomer dissolves in exudations from the damaged part and is thus removed by being absorbed by gauze or the like.

In addition, Korean Patent Application Publication No. 10-2004-0052526 proposes a wound covering material that is a foam body made from chitosan polymer. This wound covering material is advantageous in that it is not water-soluble and has high permeability but is disadvantageous in that the pores of the foam body are large and irregular in size, making the wound covering material vulnerable to intrusion of germs.

As described above, concerning the conventional wound covering materials, development and research has been focused on substances of covering materials and modification of the structure of the covering materials to improve treatment effect on wounds. That is, development and research has disregarded or ignored the principle of electrical or physical stimulation to the wounds in combination with the wound treatment effect of the covering material itself.

### SUMMARY

Accordingly, the present invention has been made keeping in mind the above problems occurring in the prior art, and an object of the present invention is to provide a medical device to improve the treatment effect on a damaged part of a living body by using electrical stimulation in combination with the treatment effect of a covering material.

Another object of the present invention is to provide a medical device having an electrical stimulator provided with a covering material.

A further object of the present invention is to provide a medical device which allows the state of a covering material in an electrical stimulator to be checked from outside.

In order to accomplish the above objects, the present invention provides a medical device having a covering material and an electrical stimulator. In one aspect, the present invention provides a medical device having a covering material and an electrical stimulator. The electrical stimulator includes a main body which generates electrical stimulation and a pad that is detachably arranged at a damaged part of a living body and that transfers the electrical stimulation to the damaged part. The covering material is disposed on a lower surface of the pad and comes into contact with the damaged part to protect and treat the damaged part.

The electrical stimulator may further include an observation window which is disposed on an upper surface of the pad and allows the state of the covering material disposed on the lower surface of the pad to be checked.

The pad may include a pad body made of an insulating material, a through hole which extends through the pad body, and an electrode which is combined with the pad body in order to transfer the electrical stimulation generated by the main body of the electrical stimulator to the living body, in which the covering material is disposed in the through hole and the observation window may be a transparent film disposed on an upper surface of the covering material.

The main body of the electrical stimulator may further include a stimulation signal generation unit to generate the electrical stimulation, and the electrode in the pad may be electrically connected to the stimulation signal generation unit via an electric wire.

The main body of the electrical stimulator may further include a simulation signal generation unit which generates the electrical stimulation and an output terminal which is connected to the stimulation signal generation unit and disposed on an outer peripheral surface of the main body, and the pad may further include a terminal connector which is connected to the electrode, is exposed from the upper surface of the pad body and is detachably connected to the output terminal.

The pad may further include a conductive adhesive layer disposed on a lower surface of the electrode.

The electrical stimulation generated by the electrical stimulator and transferred to the living body may be a direct current of 100 μA to 1 mA, a current of 10 mA with 100 pulse repetitions per second, or a high-voltage pulsed current.

The present invention provides a medical device which can improve a treatment effect on a damaged part of a living body by combining the treatment effect of a covering material and the treatment effect of electrical stimulation.

The present invention provides a medical device in which an electrical stimulator and a covering material are integrated.

The present invention provides a medical device which allows the state of a covering material in an electrical stimulator to be checked from outside.

### BRIEF DESCRIPTION OF THE DRAWINGS

FIGS. 1(a) and 1(b) are exploded perspective views of a medical device having a covering material and an electrical stimulator.

FIG. 2 is a diagram illustrating elements of the medical device illustrated in FIG. 1(a).

FIG. 3 is a diagram illustrating elements of the medical device illustrated in FIG. 1(b).

### DETAILED DESCRIPTION

The exemplary embodiment of the present invention will be described below with reference to the accompanying drawings.

Unless otherwise defined, all terms including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Structure and control methods of a medical device disclosed herein are only for illustrative purposes of the embodiments of the present invention and are not intended to limit the scope of the present invention. Like reference numerals used throughout the description herein represent like elements.

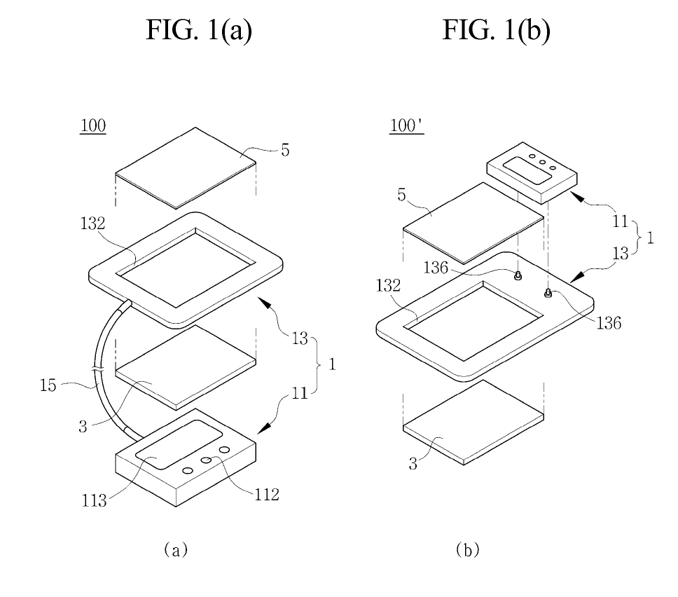

FIGS. 1(a) and 1(b) illustrate different embodiments of a medical device 100, i.e., medical device having a covering material and an electrical stimulator according to the present invention. The structure of the medical device 100 illustrated in FIG. 1(a) is described first and a description about the structure of the medical device illustrated in FIG. 1(b) follows thereafter.

The medical device 100 illustrated in FIG. 1(a) includes an electrical stimulator 1 which electrically stimulates a living body such as a human body, and a covering material 3 which is disposed in the electrical stimulator and which covers and treats a wound, or damaged tissue, of the living body.

The electrical stimulator 1 includes a main body 11 which causes electrical stimulation and a pad 13 which transfers the electrical stimulation caused by the main body 11 to the damaged part of the living body via an electric wire 15.

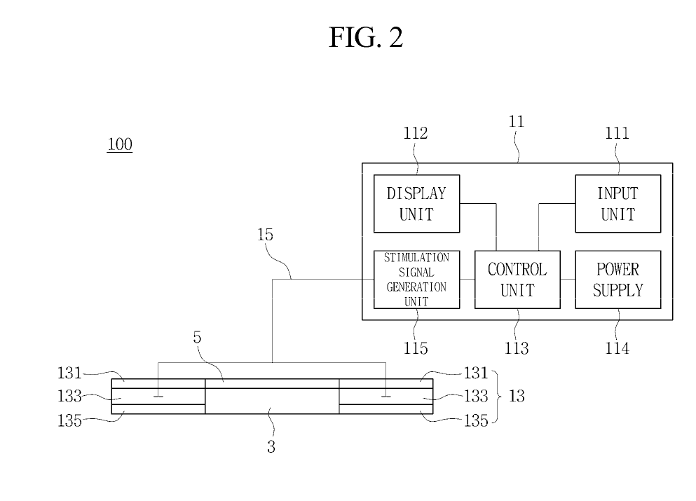

The structure of the main body 11 will be described below with reference to FIG. 2. The main body 11 of the electrical stimulator includes an input unit 111 which receives control commands from a user, a display unit 112 which displays the control command which is entered into the input unit or a history of controls on the electrical stimulator, a stimulation signal generation unit 115 which causes the electrical stimulation, a power supply 114, and a control unit 113 which controls occurrence of the electrical stimulation by the stimulation signal generation unit 114 and the magnitude of the stimulation signal.

Preferably, the pad 13 includes a pad body 131 made of an insulating material, an electrode 133 disposed on a lower surface of the pad body 131, and an adhesive layer 135 which is made of a conductive material, combined with the electrode 133, and comes into contact with the skin of a human body.

The pad 13 may further includes the covering material 3 which covers the damaged part of a living body to protect and treat the damaged part.

The covering material 3 may be a conventional wound dressing which protects a wound to prevent contamination of the wound, absorbs exudations exuded from the wound, cleans the wound, and removes impurities from the wound.

When using the medical device according to the present invention, the covering material 3 covering the damaged part of a living body treats and protects the wound from contaminants, and the electrical stimulator 1 promotes healing of the wound by transferring the electrical stimulation caused by the stimulation signal generation unit 115 to the electrode 133 via the electric wire 15 and to the living body via the adhesive layer 135.

Preferably, the electrical stimulation applied by the electrical stimulator 1 to the damaged part is a low-intensity direct current, a low-intensity pulsed current, or a high-voltage pulsed current.

When the low-intensity direct current is applied to the damaged part, healing of the damaged part is sped up two times faster than that in the case of not applying electrical stimulation to the damaged part. In this case, an example of the low-intensity direct current may be a direct current of 100 μA to 1 mA.

The pulsed current is a current which flows in one direction or switches its direction and which is periodically intermittent. The pulsed current is an intermittent current with a pulse period of several ms. It is anticipated that the treatment effect on the damaged part is highest when the low-intensity pulsed current is a current of 10 mA and its pulse is repeated 100 times per second.

The high-voltage pulsed current is a monophasic pulsed current obtained by synthesizing two phases into a single pulse to form twin-peak waves. In the high-voltage pulsed current, the pulse ratio between the pulse duration and the interpulse interval is 1:99. That is, the pulse duration is very short, allowing only a small amount of charge to accumulate in the tissue and enabling the charge accumulated in the tissue to be neutralized immediately. The average current over the pulse duration is 1.5 mA to 2.0 mA. Accordingly, the high-voltage pulsed current provides softer stimulation than the other types of current and has low skin resistance. For this reason, the high-voltage pulsed current can stimulate the damaged part without damaging the tissue.

The electrical stimulation applied by the electrical stimulator 1 to the damaged part has physiological, thermal, and chemical effects. The physiological effects include bactericidal effects, fluid shift out of area, and pain control, and the thermal and chemical effects include fast tissue healing.

The bacteriocidal effect results in retardation of the growth of bacteria. The fluid shift out of area results in relief of edema due to recoil of fluids in the vicinity of the edema when an area adjacent to the edema is stimulated using a negative electrode serving as an active electrode because blood cells and plasma proteins such as albumin are negatively charged under normal pH condition of blood.

The pain control effect is based on various mechanisms. For example, one mechanism which accounts for the pain control is promotion of secretion of neurotransmitters such as endorphins and enkephalins. Another mechanism accounting for the pain control is interception of electromagnetic waves of action potential by electrical stimulation, resulting in nerve blocking.

The effect of fast tissue healing among the thermal and chemical effects by the electrical stimulation means is obtained based on the mechanism in which electrical stimulation recovers potential loss, difference in potential between damaged tissue part and normal tissue, and promotes microcirculation and circulation of lymph in the vicinity of the damaged tissue.

Furthermore, the electrical stimulation applied to the damaged part has a vascular reflex effect which facilitates blood flow.

Accordingly, when electrical stimulation using a low-intensity direct current, a low-intensity pulsed current, or a high-voltage pulsed current is applied to the damaged part of a living body while the covering material 3 is applied to the damaged part of the living body, healing of the damaged part is expedited.

The covering material 3 and the pad 13 may be combined in various ways. The simplest structure is the form in which the covering material 3 is attached to the lower surface of the adhesive layer 135.

The covering material 3 may be smaller than the adhesive layer 135 so as not to be exposed to the outside by protruding from the adhesive layer 135 when the covering material 3 is attached to the lower surface of the adhesive layer 135. This is to enable the covering material 3 to be attached to the damaged part (wound) instead of the adhesive layer 135.

Alternatively, the covering material may be inserted in a recess (not shown) formed in the adhesive layer 135 or inserted into a recess (not shown) formed in the electrode 133 after being passed through the adhesive layer 135. Even in this case, it is preferable that the covering material 3 be smaller than the adhesive layer 135.

Since it is necessary to check the state of the covering material 3 during the use of the medical device according to the present invention, the covering material and the pad may be combined preferably in a way described below to enable the state of the covering material to be checked from the outside during the use of the medical device according to the present invention.

In order to obtain the above-described effects, the pad may includes a pad body 131, an electrode 133, and a through hole 132 passing through an adhesive layer 135. A covering material 3 may be inserted in the through hole 132, an observation window 5 may be provided in an upper surface of the covering material 3 or a peripheral surface of the through hole 132 to allow the state of the covering material 3 to be externally checked.

The observation window 5 may be made of a transparent film.

Accordingly, the user of the medical device can check the state of the covering material 3 in the pad 13 through the observation window 5 made of a transparent film, for example.

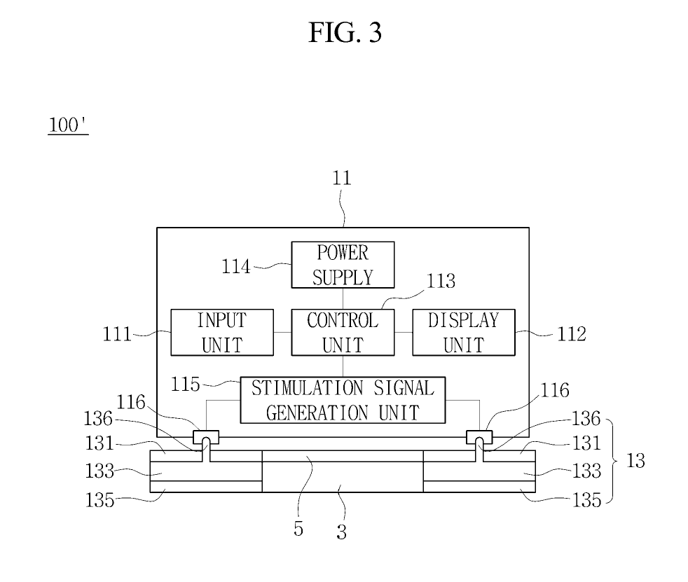

FIG. 1(b) is a diagram illustrating a structure in which the main body 11 of the electrical stimulator 1 is connected to the pad 13 without using an electric wire. The structure in which the main body 11 and the pad 13 are connected in a wireless manner will be described with reference to FIGS. 1(b) and 3.

A medical device 100′ according to the present embodiment includes an electrical stimulator 1 which applies electrical stimulation to a damaged part of a living body and a covering material 3 which comes into contact with the damaged part.

The electrical stimulator 1 in the present embodiment includes a main body 11 which generates electrical stimulation, and a pad 13 to which the main body 11 is detachably attached and which transfers the electrical stimulation generated by the main body to the damaged part of the living body.

The structure of the main body 11 will be described with reference to FIG. 3. The main body 11 of the electrical stimulator 1 includes an input unit 111 which receives control commands from a user, a display unit 112 which displays the control commands input through the input unit 111 and a control history for the electrical stimulator 1, a stimulation signal generation unit 115 which generates electrical stimulation, a power supply 114, a control unit 113 which controls generation of electrical stimulation and magnitude of a stimulation signal by the stimulation signal generation unit 115, and an output terminal 116 which is disposed in a peripheral surface of the main body 11 and is electrically connected to the stimulation signal generation unit 115.

The pad 13 includes a pad body 131 made of an insulating material, an electrode 133 disposed on a lower surface of the pad body 131, an adhesive layer 135 which is combined with the electrode 133 and is made of a conductive material and which comes into contact with the skin of a living body, and a terminal connector 136 which is detachably attached to the output terminal 116 and is connected to the electrode 133.

The terminal connector 136 is formed like a protrusion which is connected to the surface of the electrode 133 and is exposed through the upper surface of the pad body 131. The output terminal 116 is formed like a recess into which the protrusion is accommodated.

The output terminal 116 is formed like a protrusion protruding from the main body 11 to be exposed to the outside, and the terminal connector 136 may be formed like a recess into which the output terminal formed like a protrusion is accommodated.

In the medical device 100′ according to the present embodiment, since the pad 13 and the main body 11 are connected without using an electric wire, user convenience is improved.

The pad 13 is further provided with the covering material 3 which covers the damaged part of a living body, and the pad 13 and the covering material 3 may be combined into various forms.

That is, the covering material 3 may be disposed on the lower surface of the adhesive layer 135, or disposed in the recess (not shown) formed in the adhesive layer 135 or in the recess (not shown) formed in the electrode 133 after being passed through the adhesive layer 135.

When the pad 13 is provided with the pad body 131, the electrode 133, and the through hole 132 extending through the adhesive layer 135, the covering material 3 is inserted in the through hole 132, and the observation window 5 (transparent film or the like) is arranged on the upper surface of the covering material 3 and the outer peripheral surface of the through hole 132, there is an advantage that the state of the covering material 3 can be checked from the outside of the pad 13 while the medical device is being used.

The present invention provides the medical device which allows the state of the covering material to be checked from outside as well as the medical device 100 or 100′ which promotes treatment of a wound using electrical stimulation in combination with the covering material.

Since the present invention can be embodied in various forms, the scope of the invention is not limited to the above embodiments. That is, as long as a structure includes the essential elements of the appended claims, the structure falls under the scope of the invention.

1. A medical device, comprising:
an electric stimulator comprising a main body to generate electric stimulation, and a pad detachably arranged at a damaged part of a living body so as to provide the damaged part of the living body with the electric stimulation generated from the main body; and
a covering material arranged at a lower surface of the pad to protect and treat the damaged part.

2. The medical device according to claim 1, further comprising an observation window disposed in an upper surface of the pad to observe the covering material disposed on the lower surface of the pad.

3. The medical device according to claim 2, wherein the pad comprises a pad body made of an insulating material, a through hole which extends through the pad body, and an electrode which is combined with the pad body and which transfers the electrical stimulation generated by the main body to the living body;
the covering material is disposed in the through hole, and
the observation window disposed in the upper surface of the covering material is a transparent film.

4. The medical device according to claim 3, wherein the main body further includes a stimulation signal generation unit which generates the electrical stimulation; and
the electrode provided in the pad is connected to the stimulation signal generation unit via an electric wire.

5. The medical device according to claim 3, wherein the main body further includes a stimulation signal generation unit to generate electrical stimulation and an output terminal which is connected to the stimulation signal generation unit and which is disposed on an outer peripheral surface of the main body; and
the pad further includes a terminal connector which is connected to the electrode and is exposed from an upper surface of the pad body and which is detachably connected to the output terminal.

6. The medical device according to claim 4, wherein the pad includes a conductive adhesive layer disposed on a lower surface of the electrode.

7. The medical device according to claim 1, wherein the electrical stimulation applied by the electrical stimulator to the living body is a direct current of 100 μA to 1 mA.

8. The medical device according to claim 1, wherein the electrical stimulation applied by the electrical stimulator to the living body is a current of 10 mA having 100 times of pulse repetition per second.

9. The medical device according to claim 1, wherein the electrical stimulation applied by the electrical stimulator to the living body is a high-voltage pulsed current.

10. The medical device according to claim 5, wherein the pad includes a conductive adhesive layer disposed on a lower surface of the electrode.
